(19) United States
(12) Patent Application Publication
(10) Pub. No.: US 2006/0166268 A1
(43) Pub. Date: Jul. 27, 2006

(54) Diagnosis of glaucoma by complex autoantibody repertoires in body fluids

(75) Inventors: Franz Grus, Mainz (DE); Stephanie Joachim, Bodolz (DE); Norbert Pfeiffer, Mainz (DE)

Correspondence Address: WENDEROTH, LIND & PONACK, L.L.P., 2033 K STREET N. W., SUITE 800, WASHINGTON, DC, 20006-1021, US

(73) Assignee: RESCOM GMBH, Baar (CH)

(21) Appl. No.: 10/531,385

(22) PCT Filed: Oct. 16, 2003
(86) PCT No.: PCT/IB03/04597

§ 371 Date: May 16, 2005

(30) Foreign Application Priority Data
Oct. 18, 2002 (EP) 02023332.6

(51) Int. Cl. G01N 33/53

(52) U.S. Cl. 435/7.1

(57) ABSTRACT

The invention relates to a method for the diagnosis of glaucoma based on the composition of autoantibodies against ocular antigens in body fluids of individuals. The method is characterized in that in a first step, autoantibodies against retinal and/or optic nerve head antigens are detected and measured in body fluids of an individual, and, in a second step, the auto-antibody pattern is correlated with corresponding patterns of healthy individuals and glaucoma patients. The invention further relates to a method for assessing an individual's risk for developing glaucoma, and to kits for use in the method of the invention.

### BACKGROUND OF THE INVENTION

Glaucoma, one of the leading causes of blindness worldwide [1], represents a group of ocular disorders that are responsible for loss of retinal ganglion cells and their axons, damage to the optic nerve, and gradual loss of visual field. People of all age-groups can be affected by this disease. At the age of 70 about seven percent of the population are suffering from glaucoma. Unlike other eye disorders, the causes of glaucoma and the best way to treat them are still not completely investigated. The optic nerve head is considered the earliest site of nerve damage in glaucoma. The damage of these nerve fibers causes retinal ganglion cell death through retrograde degeneration. This can lead to total and irreversible blindness.

Although elevated intraocular pressure can be an important risk factor of glaucoma, it is no longer considered as an essential part of the definition of the disease, since some glaucomatous patients have normal intraocular pressures [2]. Only 76% of the patients with glaucoma have an intraocular pressure higher than 21 mm Hg [3]. Intraocular pressure of about 16% of glaucomatous eyes was never recorded above 21 mm Hg. Thus, a more effective diagnostic criterion is necessary.

Currently, the most important screening methods for glaucomatous patients include tonometry, ophthalmoscopy and perimetry. Despite its well-known limitations, measuring the intraocular pressure (tonometry) is the most common method for glaucoma screening. Tonometry has a sensitivity and specificity of about 65 percent. Ophthalmoscopy is a method for examining optic nerve head morphology. But the usefulness of this method is highly dependent on the skills of the examiner. Perimetry permits diagnosis of visual field loss, which is an indicator for ganglion cell death. But this method allows only detecting severe damage at an advanced stage of the disease. Therefore, a diagnostic method is needed which is more objective and more sensitive and can be applied at an earlier stage in the evolution of the disease (i.e. before irreversible damage is manifest).

There are different kinds of glaucoma. Because the pathophysiology, clinical presence and treatment of the different types of glaucoma are so varied, there is no single definition that adequately encompasses all forms. The most common form is primary open-angle glaucoma (POAG), characterized by optic nerve damage and high intraocular pressure. But there is also a high number of patients who never had elevated intraocular pressure; this form of glaucoma is usually called normal tension glaucoma (NTG). There are several other forms of primary and secondary glaucoma. There is not only adult onset glaucoma but there are also juvenile forms of glaucoma.

In some patients there is evidence for an autoimmune mechanism being responsible for glaucomatous injury to the optic nerve. Their glaucoma constitutes an organ-specific autoimmune disease, characterized by immune-mediated tissue destruction occurring in a limited range of tissue types or cells [4].

Several specific antibodies have been detected in serum samples of glaucomatous patients.

The different families of heat shock proteins (HSPs) were detected in sera of patients with glaucoma like POAG or NTG. Heat shock proteins can be divided into different groups on the basis of their molecular weight, such as HSP-90 (90 kilo Dalton), HSP-70 (70 kDa), HSP-60 (60 kDa), and small heat shock proteins (25 to 30 kDa). Heat shock proteins, also called stress proteins, serve as cellular chaperones [5] and as protectors for the whole organism. Heat shock proteins appear everywhere in normal tissue. By carrying immunogenic peptides, they are capable of triggering an immune response. They are considered neuroprotective and are expressed as a response to stress conditions such as ischemia or excitotoxicity. But people with reduced or elevated levels of heat shock proteins could be lacking this neuroprotective factor. This deficiency might be a possible cause for the development of glaucoma. High titers of autoantibodies to heat shock proteins could lead to optic neuropathy in these patients [6]. In patients with glaucoma, elevated serum autoantibodies against small heat shock proteins have been found [7]. These small heat shock proteins include α-crystallins (αA-crystallin and αB-crystallin subunits) and HSP-27. α-Crystallin is the predominant structural protein of the ocular lens and is also found in retinal cells. Hsp-27 antibody can enter retinal cells and this can lead to apoptosis of these cells [8].

No association between the serum HSP antibody titers and optic disc parameters or peripapillary atrophy parameters has been found [9].

Wax et al. developed an animal model to back up the thesis that autoantibodies can be a cause of glaucoma [10]. Rats were immunized with Hsp27 and showed a degeneration of neurons in the retinal ganglion cell layer.

Antibodies against human vimentin and human glial fibrillary acid protein (GFAP) were detected in human optic nerve astrocytes exposed to elevated hydrostatic pressure [11]. The astrocytes developed an increased synthesis and redistribution of vimentin and GFAP.

Anti-Ro/SS-A (Sjögren syndrome A; also commonly called Ro antigens) antibodies were detected in patients with normal tension glaucoma [12].

Romano et al. tested sera from glaucoma patients for anti-rhodopsin antibodies against retinal antigens [13]. Those patients had a priori clinically diagnosed POAG or NTG. Western blots of these sera showed antibodies against a 40-kDa antigen which was later identified as rhodopsin.

Serum antibodies that bind to human optic nerve head proteoglycans, including chondroitin sulfate and heparin, are found in patients with glaucoma [14]. Glycosaminoglycans (GAGs) play an important role as membrane proteins; they are components of extracellular matrix in the optic nerve head.

Serum autoantibodies against gamma-enolase (γ-enolase) in retinal ganglion cells of glaucomatous patients have been detected by Maruyama et al. [15]. These antibody levels were elevated in patients with glaucoma and were found in approximately 20% of glaucomatous patients. γ-Enolase is a neuron-specific enolase.

Lymphocytes in the blood of glaucoma patients were examined for antigens by Yang et al. These analyses showed that sera from patients with glaucoma have elevated small interleukin-2 (sII-2) levels [16]. sII-2 is produced by T-cells and increases the level of other cytokines and antibodies.

Glutathione S-transferase (GST) antibodies were detected in glaucomatous patient sera [17]. These antibodies bound against bovine retina. GST is expressed in tissue cytosols and membranes. It is present in the central nervous system, the retina, and throughout the whole body.

Tumor necrosis factor (TNF)-α and TNF-α receptor-1 are elevated in the retina of glaucoma eyes compared to healthy donors [18]. Tezel et al. found TNF-α in glaucomatous eye was more intense in retinal areas close to the optic nerve head than in the periphery. TNF-α production is increased in retinal glial cells after exposure to elevated hydrostatic pressure or insulted ischemia [19]. High TNF-α levels lead to apoptosis by driving axonal degeneration [20]. Methods for treating glaucoma by inhibiting TNF-α are subject of patent application WO 01/58469.

Antiphosphatidylserine (APS) antibodies have been detected in patients with normal tension glaucoma [21]. The NTG patients showed higher levels than healthy control subjects and patients with primary open angle glaucoma. Antiphosphatidylserine antibodies are a subspecies of antiphospholipid antibodies (APL) and bind to phosphatidylserine molecules. Other subspecies are cardiolipin (ACL) and β2-glycoprotein (β2GP). Through this cascade the antibodies may be responsible for causing thrombosis. Cardiolipin binds to apoptotic cells which leads to an increased level of tumor necrosis factor-α (TNF-α). Therefore, they play an important role in thrombosis development.

Myocilin/TIGR is expressed in optic nerves and trabecular meshwork of glaucomatous eyes [22]. TIGR is the former name of myocilin. The researchers observed a loss of myocilin in glaucomatous eyes. Its expression in the trabecular meshwork is thought to be responsible for the elevated intraocular pressure associated with some forms of glaucoma [23]. Mutations of the TIGR gene increase the risk of early-onset glaucoma. The presence of the apolipoprotein E (ApoE) allele is an indicative of an increased risk of developing early-onset glaucoma, see patent application WO 00/68429.

A human transcription factor named FKHL7 is expressed most abundantly in the eye. Patent application WO 99/53060 describes this factor and its use in treating a diagnosing glaucoma

Optineurin antibody has been detected in whole-cell extracts from patients with adult-onset primary open-angle glaucoma [24]. Optineurin may be part of the tumor necrosis factor-α signaling pathway. It is expressed in trabecular meshwork, ciliary epithelium, and retina

γ-Synuclein proteins occur in the optic nerve of glaucomatous eyes in a significantly higher level than in control eyes without glaucoma [25]. Synucleins contribute to the pathology of neuronal degeneration. All three members of synucleins (α-, β-, and γ-synuclein) are expressed in retina and optic nerve. The bovine orthologue of γ-synuclein, synoretin, is mainly localized to the nuclear and synaptic regions of retinal cells [26].

Testing for serum autoantibodies in the clinical evaluation of neuropathy syndromes is widely practiced. Autoantibodies can serve as markers in diagnosis and can lead to prognosis and treatment. Although some auto-antibodies in the sera of glaucoma patients have been identified and correlated with the glaucoma disease, as described hereinbefore, many still remain unknown.

Western blotting has surfaced as a powerful tool for detecting specific autoantibodies in immune diseases. Complicating the straightforward identification of pathogenically relevant antigens, however, is that normal sera contain large amounts of natural antibodies which manifest themselves in complex staining patterns [27][28]. Thus, it can complicate the differentiation of disease-associated auto-antibodies from the complex background of “auto-immune noise”, i.e. naturally occurring autoantibodies. Most of previous studies evaluated one or a few specific disease-related antibodies and have screened only a limited number of purified homologous or heterologous proteins as antigens by means of ELISA or RIA. A diagnosis based on these antibodies was impossible to establish. On the other hand Western blotting has evolved as the most important tool to demonstrate autoantibodies in auto-immune diseases because it permits simultaneous screening for a wide spectrum of different auto-antigens. A recent new technique, capable of analyzing these complex staining patterns of Western blots simultaneously, is based on digital image analysis. This technique has been successfully used in studies of myasthenia gravis, Graves' disease and experimental uveitis [29][30][31].

### SUMMARY OF THE INVENTION

The invention relates to a method for the diagnosis of glaucoma based on the composition of autoantibodies against ocular antigens in body fluids of an individual. The method is characterized in that in a first step, autoantibodies against ocular antigens are detected and measured in body fluids of an individual, and, in a second step, the autoantibody pattern is correlated with corresponding patterns of healthy individuals and glaucoma patients.

The invention further relates to a method for assessing an individual's risk for developing glaucoma with or without an increased intraocular pressure by analyzing the autoantibody repertoire in the individual's body fluids against ocular antigens as biomarkers for the diagnosis of glaucoma.

The invention further relates to kits for use in the method of the invention.

### DETAILED DESCRIPTION OF THE INVENTION

The invention relates to a method for the diagnosis of glaucoma based on the composition of autoantibodies in body fluids of patients. The term “glaucoma”, as used herein, refers to all kind of primary open-angle glaucoma, including both juvenile-onset and adult- or late-onset glaucoma, pseudoexfoliation (PEX) syndrome and PEX glaucoma, with or without an elevated intraocular pressure, and to normal pressure glaucoma (normal tension glaucoma, NTG, or low-tension glaucoma, LTG).

In the method of the invention, the complex autoantibody repertoires in body fluids of patients are used to set up a diagnosis and prognosis for the development and course of glaucoma. In the first step, the method detects the autoantibodies against ocular antigens, e.g. against retinal antigens, optic nerve antigens, optic nerve head antigens, trabecular meshwork antigens, uveal antigens, or a mixture of them, in particular retinal antigens and optic nerve head antigens, in body fluids of an individual. “Body fluids” includes all fluids containing antibodies such as sera, tears, saliva, urine, aqueous humour, vitreous body of the eye, etc. Preferred body fluids in the method of the invention are sera and tears, in particular sera. In the second step, the autoantibody pattern is correlated with corresponding patterns of healthy individuals and glaucoma patients, in particular patients with different types of glaucoma. The invention also relates to the method of comparison of complex autoantibody patterns by calculation, e.g. a method of comparison of complex autoantibody patterns by calculation wherein a pattern of autoantibodies against ocular antigens of an individual is compared with the corresponding autoantibody pattern of healthy individuals and of glaucoma patients, e.g. patients with primary open-angle glaucoma or patients with normal tension glaucoma.

It may be noted that the invention relates to a comparison of an autoantibody pattern, i.e. a complex mixture of a large number of autoantibodies both known (and discussed in the background section) and unknown. Under “large number” at least 10 autoantibodies, more preferably at least 20, most preferably at least 30 autoantibodies are understood. It is of no relevance for the method of the invention whether the particular antibody or the antigens are properly characterized, since the procedure relies only on a molecular mass comparison.

Detection of autoantibody pattern may be by conventional Western blot techniques. However, the method of the invention also includes the use of other commercially available or experimental detection techniques such as chemiluminescence assays, ELISA, RIA, and techniques based on microarray chips, such as SELDI-TOF-type (surface-enhanced laser desorption/ionization in time-of-flight mass spectrometry; available e.g. under the trade name ProteinChip™ Array, from Ciphergen, Fremont, Calif., USA), matrix assisted laser desorption/ionization (MALDI) mass spectroscopy, or other antibody-based chip array techniques (e.g. from BD Biosciences Clontech, Heidelberg, Germany).

The SELDI-TOF technique allows mass screening for auto-antibodies in a very reliable, fast, and extremely sensitive manner. For example, the ProteinChip™ system (Ciphergen) uses protein chip arrays and SELDI-TOF technology for capturing, detection, and analysis of proteins without labelling and without the need for chemical modification. The micro-scale design of the arrays allows the analysis of very small quantities of proteins. Arrays with biologically activated surfaces are used that permit antibody capture studies. Preferably, protein-A chips are incubated with sera of patients, then treated with a complex solution of auto-antigens, i.e. ocular antigens. If the protein-A bound autoantibodies recognize their antigens, these proteins can be separated by their molecular masses and detected by mass spectrometry. At higher molecular weights (>30 kDa) the detection sensitivity of this on-chip method is comparable to conventional Western blotting. At lower molecular weights, the sensitivity of the Western blot technique is easily surpassed by the on-chip method. The on-chip procedure is easy to use, less time consuming than Western blotting, and more sensitive at least in the low molecular weight range. Furthermore, the antigen-antibody reactions can be performed using beads binding autoantibodies. After elution of the antigens bound by antigen-antibody reactions from the beads, they can be analyzed using SELDI-TOF chips or conventional electrophoretical techniques.

Antibody-based chip arrays (e.g. Clontech) facilitate the diagnosis process by just spotting onto a micro-chip appropriate ocular antigens that subsequently are recognized by their antibodies in the sera of patients.

After detection, the complex autoantibody repertoires of body fluids are read in a digital image analysis system or other device for digitization, and subsequently analyzed by multivariate statistical techniques, e.g. analysis of discriminance, classification/regression trees, and/or artificial neural networks. Artificial neural networks learn from experience, not from programming. They are fast, tolerant of imperfect data, and do not need formulas or rules. Artificial neural networks are able to generalize and extract consistent features of patterns used to train them. In the present invention, the artificial neural network is “trained” to recognize the antibody patterns of glaucoma patients. The preferred artificial neural network used in the invention is the multiple layer feedforward network (MLFN) with the backpropagation training algorithm. This kind of network is typically formed by three layers: The input layer receives information from the “external world”. The output layer presents the results to the connected devices. A layer of hidden neurons is sandwiched between them. Networks are trained by presenting known samples to them. The network attempts to change the function (weight) of each neuron until all training samples are classified correctly.

Other types of artificial neural networks may be used, e.g. self-propagation procedures, probalistic neural networks, other kind of training algorithms, pruning techniques, and genetic algorithms.

These data analysis techniques differentiate between healthy individuals and glaucoma patients by considering the whole complex antibody pattern of each patient. By comparing the antibody pattern of a patient with suspected glaucoma with the antibody pattern in samples of other glaucoma patients and healthy individuals, the method can calculate to which clinical group the autoantibody pattern of a patient with unknown pathology reveals the highest similarity.

The method of the invention not only includes the computational techniques as demonstrated herein, but also similar technologies, e.g. the use of other pattern matching techniques, other classifying statistical techniques or other methods to acquire the antibody-antigen reaction data of individuals.

In this complex antibody repertoire used, some of the antigen-antibody reactions are already known and described by others, as described in the background section. Further antibody-antigen interactions analyzed with the method of the invention are still unknown.

Several antigen-antibody reactions are significantly higher in healthy individuals than in glaucoma patients. Reactivities which are absent in glaucoma patients are also used in the analysis of the antibody repertoires according to this invention. They contribute to higher sensitivity and specificity of the diagnostic method. The use of autoantibodies that are not present or have or lower reactivity in auto-immune patients compared to controls for diagnosing auto-immune diseases is also part of this invention.

The method of this invention uses the increase or decrease of autoantibodies in diseased patients, compared to controls, as “biomarkers” for the diagnosis of the disease. This implies that knowledge about the identity of those antibodies is not needed for a reliable diagnosis of the disease. No single autoantibody reliably distinguishes glaucoma patients from healthy individuals.

Although the method is described for the diagnosis of glaucoma patients, it can also be used for the assessment of a (healthy) individual's risk for developing glaucoma with or without an elevated intraocular pressure. Furthermore, the described method is useful for assessment of progression and/or severeness of glaucoma. For that purpose the change in the antibody pattern over time and corresponding computational and pattern matching techniques are used.

Kits for diagnosis of glaucoma according to the invention are based on conventional Western blotting technique, on protein chip approaches or other techniques useful in the method of this invention. A kit may comprise a ready-to-use antigen mixture, chemicals and materials needed to perform the biochemical analysis, e.g. Western blots or biochips, and, optionally, a bundled computer software including the specialized algorithm to detect glaucoma in the antibody pattern of patients. Kits according to the invention may be for use by a doctor or even a patient on its own, or for a professional diagnostic service laboratory center.

While this invention has been particularly shown and described with references to preferred embodiments thereof, it will be understood by those skilled in the art that various changes in form and details may be made therein without departing from the spirit and scope of the invention as defined by the appended claims.

### DESCRIPTION OF THE FIGURES

FIG. 1: Antigen-antibody reactions from Western blots of several patients. The intensity of staining reaction of a single Western blot (z-axis, arbitrary scanner units U) is plotted vs. the molecular weight (x-axis, kilo-Daltons kDa) and the patient (y-axis). Patients belong to the clinical groups primary open-angle glaucoma (POAG), normal tension glaucoma (NTG), and healthy subjects (CTRL).

FIG. 2: Antigen-antibody reactions against retinal antigens from Western blots plotted (Scanner units U) vs. their molecular weight (kilo-Daltons kDa, x-axis).

1: healthy control subjects (CTRL); 2: patients with primary open-angle glaucoma (POAG); 3: patients with normal tension glaucoma (NTG).

1: healthy control subjects (CTRL); 2: patients with primary open-angle glaucoma (POAG); 3: patients with normal tension glaucoma (NTG).

FIG. 3: Antigen-antibody reactions against optic nerve head antigens from Western blots plotted (Scanner units U) vs. their molecular weight (kilo-Daltons kDa, x-axis).

1: healthy control subjects (CTRL); 2: patients with primary open-angle glaucoma (POAG); 3: patients with normal tension glaucoma (NTG).

FIG. 4: ROC (responder operating characteristic) scatter plot (true-positive vs. false positive) for the diagnosis of glaucoma by means of complex auto-antibody repertoires. x-axis: specificity, y-axis: sensitivity. The calculation of the area under curve (AUC) results in an r-value of 0.92 (which is a very good diagnostic test).

FIG. 5: Analysis of antigen-antibody reactions in patients with primary open-angle and normal tension glaucoma (GL) and healthy subjects (CTRL) based on protein-chip technologies. The autoantibodies in sera of patients are captured by Ciphergen IDM-beads, incubated with ocular antigens and measured by the ProteinChip-Reader (time-of-flight (TOF) mass spectrometry). Scanner units U (y-axis) vs. molecular weight (kilo-Dalton kDa, x-axis). NULL=No antigens used in analysis sample.

### EXAMPLES

A total of 524 patients are divided into analysis groups: healthy volunteers without any ocular disorders (CTRL, n=189), patients with primary open angle glaucoma (POAG, n=96), and normal tension glaucoma (NTG, n=74). According to the classification guidelines of the European Glaucoma Society, patients suffering from ocular hypertension (OHT, n=87) without any glaucoma damage are classified as healthy controls and are included in the CTRL group in this study. To test the robustness of the glaucoma detection, in an additional procedure 165 patients with other ocular disorders (e.g. retinal diseases) are included in the non-glaucoma group (CTRL2).

The inclusion criteria for POAG are: intraocular pressure (IOP) more than 21 mm Hg, untreated, on at least one occasion. No known alternative reason for elevated IOP like alternative causes of optic neuropathy (e.g. infection, inflammation, ischemic disease, and compressive lesions). Intraocular pressure is determined by Goldmann applanation tonometer, and visual field is examined by Goldmann perimeter.

Criteria for OHT: no optic nerve cupping or visual field loss, but elevated IOP (=more than 23 mm Hg), and also open angles and the absence of alternative causes of optic neuropathy.

CTRL group criteria: healthy subjects with no history of ocular disorders, no pathologic fundus, no elevated IOP, and no eye medication.

Exclusion criteria for all groups: acute attack of glaucoma, diabetes mellitus and retinopathy, retinal detachment, and retinal vascular obliteration.

All groups are matched for age and gender. After giving their informed consent blood is taken from all patients. Those samples are centrifuged and the serum stored for later examination.

Western Blots

The sera of patients are tested against Western blots of retinal and optic nerve antigens.

Retinas from bovine eyes are dissected. They are homogenized in sample buffer (1 M Tris, pH 7.5; 10% SDS; DTT; bromophenol blue, pH 6.8), and separated by centrifugation (15000 rpm for one hour). The samples are cooked and homogenized several times. The pellet is discarded and the supernatant is stored for later analysis.

The retina extracts are used for 13.5% sodium dodecyl sulfate polyacrylamide gel electrophoresis (SDS-PAGE) using MultiGel-Long (Biometra, Goettingen, Germany). After electrophoresis the gels are transferred to nitrocellulose membrane Protran BA 83 (Schleicher and Schuell, Dassel, Germany) by using a Semi-Dry Blotter (Biometra, Goettingen, Germany). After blotting the membranes for one hour, the quality of the transfer is checked by staining the nitrocellulose with Ponceau S solution (Sigma, Munich, Germany). The blots are blocked with blocking buffer (5% non-fat dry milk in phosphate-buffered saline (PBS)) for one hour. The nitrocellulose is cut into 4 mm wide strips. One strip is used per patient. The strips are incubated over night with patient serum (1:25 dilution, in washing buffer). 1000 μl volume per strip is used. A negative control strip (as negative control one strip is incubated only with wash buffer and no serum) and a positive control strip (a sample with known antibody reactivity) are done for each blot to check the quality of the immunostaining. After washing the strips with Tris-buffered saline (TBS) for several times, they are incubated with secondary antibody (peroxidase-conjugated Immuno Pure® Goat Anti-Human IgG (H+ L); diluted 1:500, Pierce, Ill., USA) for one hour. After several washing steps the bands are developed by 0.05% 4-chloro-1-naphthol (Sigma, Munich, Germany) with 0.015% hydrogen peroxide in 20% methanol in TBS for 20 minutes.

Molecular weights are estimated for each band based on the distance migrated for ten known molecular weight standards (BenchMark, Invitrogen, Karlsruhe, Germany).

Digital image analysis and evaluation of the densitometric data of the electrophoretic separations are performed by means of the BioDocAnalyze™ software package (Biometra, Goettingen, Germany). BioDocAnalyze™ creates densitometric data files for each Western blot, which show the gray-intensity values (8-bit gray values) versus the Rf values (relative mobility, x-axis). BioDocAnalyze™ evaluates the height, area, molecular weight, Rf value, etc. of all peaks in this densitometric data file and also includes a photographic quality half tone bitmap.

From each densitometric data file, two data vectors are built in the following way: First, the Rf axis (=molecular weight) is broken into 1000 classes such that each variable of the vector represents 1/1000 of the molecular weight region. For each molecular weight of this 1000 class data vector the corresponding grey value of the densitograph of each western blot, which represents the intensity of the antigen-antibody reaction, is calculated and normalized according to the entire area und curve. Thus, each variable of the data vector represents the percent area of the peaks of the electrophoretic lane at the corresponding Rf region.

Furthermore, another data vector with a list of all peak found in each Western blot is built. For each peak, the area under curve and the intensity is recorded. The data vectors are compiled into a database for subsequent calculations, and each of them is randomly divided in two subsets: The test (unknown data, not used in the calculation procedure) and the training set (known data, used in the calculation procedure).

Calculation Procedures

First, for all Western blots in the database the algorithms group the peaks into clusters. For each peak, which is below a given threshold, the algorithm searches in all other Western blots if at this specific molecular weight region a peak can be found. In this way, a list of peaks is built revealing for all Western blot lanes the existence (and intensity) of peaks for all molecular weight regions where in more than 10% of lanes peaks could be detected. Using this generated list of peak clusters, multivariate algorithms such as analysis of discriminance or classification trees can be performed to analyze the complex banding patterns.

Multivariate analysis of discriminance: As described above, for each Western blot data vectors are exported and peak clusters are built. Each data file is assigned to solely one of both predefined groups: “healthy” or “glaucoma”. The analysis of discriminance can test the null-hypothesis that data vectors of the groups arise from a multivariate normally-distributed population.

Artificial Neural Network (ANN): The multiple layer feedforward network (MLFN) with the backpropagation training algorithm is used as provided in the Statistica 6.0 software package (StatSoft, Tulsa, USA). All data vectors are presented to the network for the learning process.

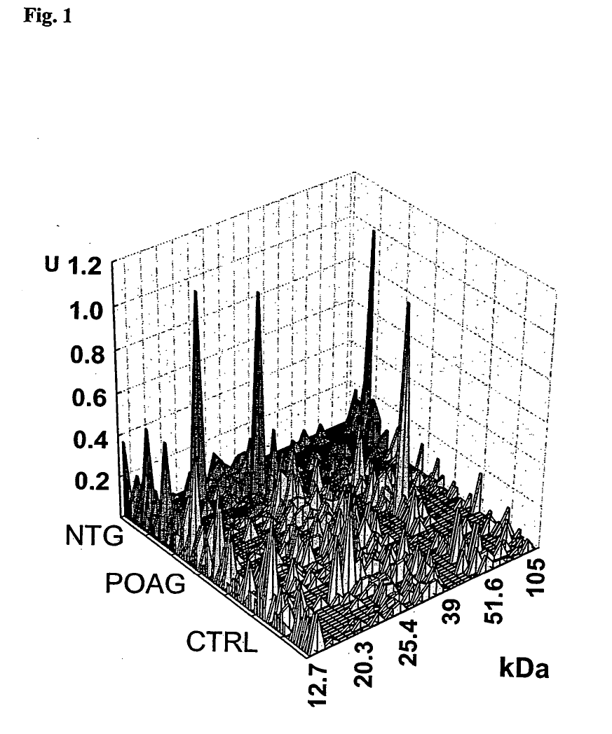

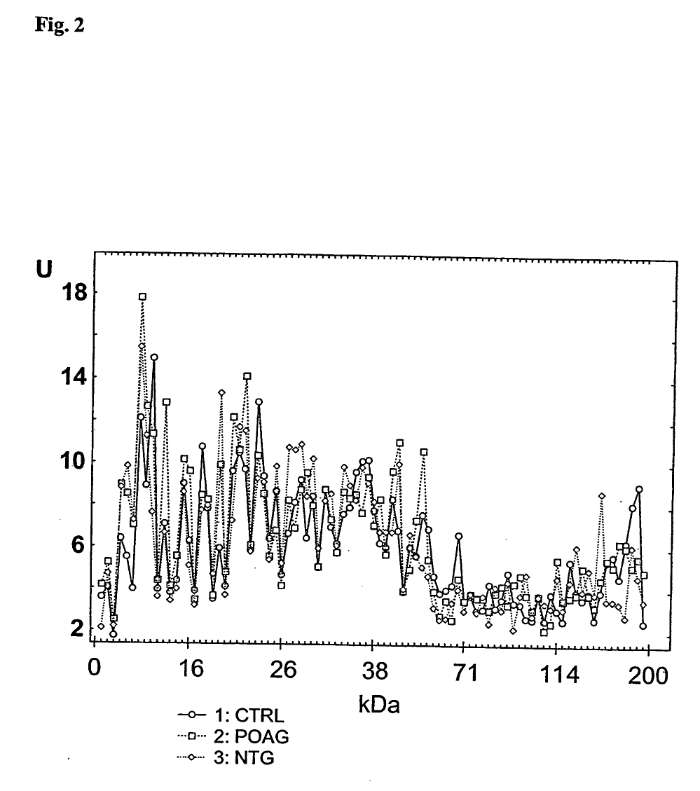

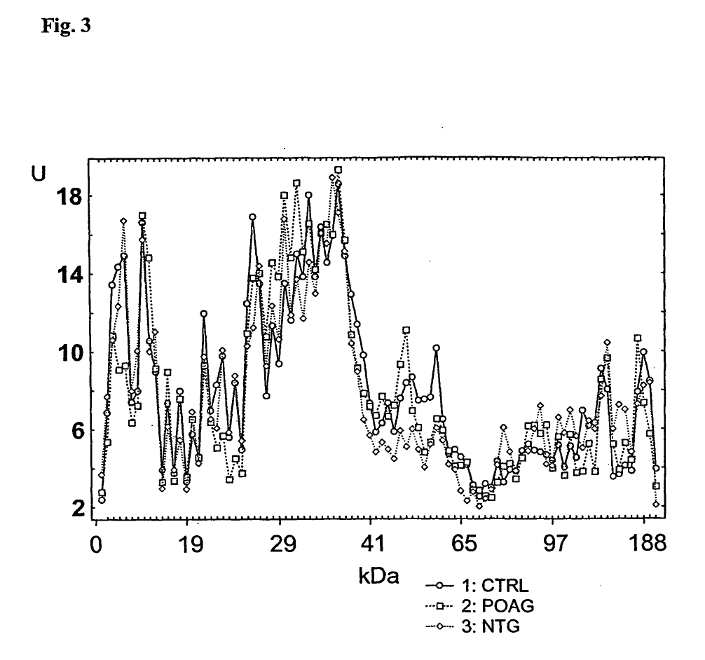

All patients exhibit different, complex staining patterns of autoantibodies against retinal antigens and optic nerve head antigens. FIG. 1 reveals the complex antibody patterns of several patients. The number of peaks of Western blots against retinal antigens is increased in sera of POAG patients compared to all other groups. Including all peaks the analysis of discriminance reveals a statistically significant difference between the patterns of POAG and NTG compared to all other groups (P<0.01). FIGS. 2 and 3 demonstrate the mean data vectors of antigen-antibody reactions for the clinical groups POAG, NTG, and controls (CTRL).

If the control group is divided into “normal” and “ocular hypertension without glaucoma (OHT)”, a statistically different antibody pattern in OHT patients compared to controls is found. Thus, the analysis of antibody pattern could not only be used to detect glaucoma, but also be useful in understanding the phenomenon of ocular hypertension using these OHT specific antibody patterns.

Furthermore, these antibody repertoires shown in FIGS. 2 and 3 are analyzed by classification procedures (multivariate statistics and artificial neural networks).

In the following the calculation procedures used in this Example is described. However, they can change slightly after inclusion of more patients or moving to other technologies than Western blotting.

Using analysis of discriminance the most important molecular weight regions are included in this classification approach. To be included, the variables must show a statistically significant difference (P<0.05) between glaucoma and no glaucoma. 40 variables are included in the approach. These variables (molecular weight regions) are used as input neurons in an artificial neural network. The three layer feedforward network (MLFN) with the backpropagation training algorithm is used. The network has one output neuron which can have two states: glaucoma or no glaucoma. The network is allowed to prune those variables that do not help to distinguish between glaucoma or no glaucoma. The data vectors of all patients are randomly divided into a test and training group. The training groups are used to setup a neural network which is optimized to find rules in the complex antibody patterns to detect glaucoma from the immunological noise of naturally occurring antibodies. The test set, which is not included during training, is used to test if the neural network is able to detect glaucoma patterns form the patient group. Because the analysis of discriminance can demonstrate that the patterns of NTG and POAG are specific and different from each other, two neural networks are built: the first one searching for NTG patterns and the second searching for POAG patterns. The output of both nets are combined to detect glaucoma patterns in general. The algorithm uses those regions of molecular weight that are found to be the most important to distinguish between the groups determined by the sensitivity analysis of artificial neural networks. The analysis includes regions of already known antibodies, regions of unknown antibodies not described before, and especially regions where the reactivities in glaucoma patients are significantly lower than in healthy subjects.

After training, the following antigen-antibody reactivities (in kDa) are included: 11.2, 18.5, 9.8, 63.5, 28.9, 193, 12.1, 114.4, 110.4, 152, 68.1, 133.6, 48, 157, 174, 19.1, 25, 27, 60.1, 197, 163, 20, 169, 7.8, 186, 105, 14, 15.6, 142, 13, 42, 26.5, 23.4, 65, 57, 21.6, 88.3, 39, 10, 36, 179.8, 78. The regions are ranked according to the importance on the decision of the neural networks.

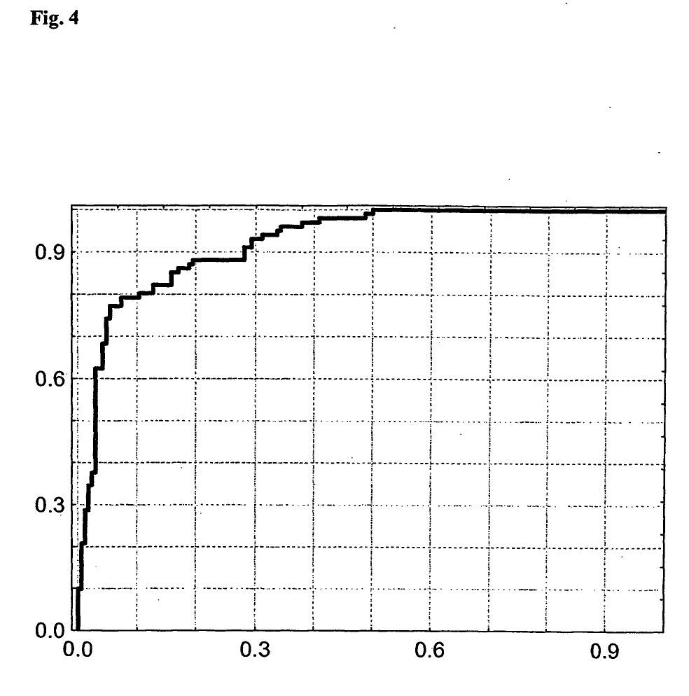

After training, the test procedure is performed using the test data set. 83.5% of glaucoma patients and 85.2% of healthy subjects are correctly classified. This is equivalent to a sensitivity of 83.5% and a specificity of 85.2%. Using antibody patterns to classify glaucoma patients, a responder operating characteristic (ROC) analysis is calculated and an r-value of approximately 0.92 is found, which is indicative of a very good diagnostic test (FIG. 4).

To test the performance of the neural network, 165 non-glaucoma patients with other ocular diseases (e.g. age dependent macular degeneration) are additionally included in the analysis (CTRL2). The inclusion of those patients with other ocular disorders slightly decreases the AUC r of the ROC curve to 0.84. Considering that now 524 patients are included in the study and the test is solely based on the antibody patterns without knowledge of any other clinical parameters of patients, this is again a very good diagnostic test.

Protein Chip

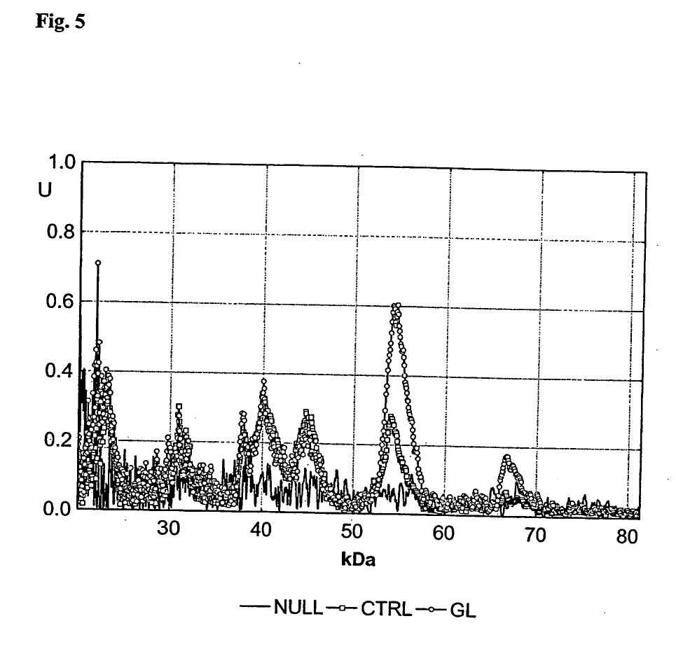

The autoantibodies in the sera of patients are coupled to Ciphergen IDM beads and subsequently incubated with ocular antigens from retina or optic nerve. After elution from the beads, the antigens can be analyzed by a time-of-flight mass spectrometer using Seldi-TOF surfaces. Using this approach, a complex pattern of antigen-antibody reactions can be found in the sera of patients. In FIG. 5, those group profiles of antibody reactions are demonstrated for some molecular weight regions for glaucoma patients (GL), healthy subjects (CTRL), and a negative control without antigens (NULL).

### REFERENCES

[1] Quigley H A, Br J Ophthalmol. 1996; 80:385-387
[2] Quigley H A, N Engl J Med. 1993; 328(15):1097-1106
[3] Sommer A, Arch Ophthalmol. 1991; 109(8):1090-1095
[4] Wax M B, Curr Opin Ophthalmol. 2000; 11(2):145-150
[5] Tezel G, Arch Ophthalmol. 2000; 118(4):511-518
[6] Wax M B, Arch Ophthalmol. 1998; 116(8):993-1001
[7] Tezel G, Invest Ophthalmol Vis Sci. 1998; 39(12):2277-2287
[8] Tezel G, J Neurosci. 2000; 20(10):3552-3562
[9] Wax M B, Ophthalmol. 2001; 108(2):296-302
[10] Wax M B. A Model of Experimental Autoimmune Glaucoma in Rats Elicited by Immunization With Heat Shock Protein 27. ARVO Abstract, 2002
[11] Salvador-Silva M, J Neurosci Re. 2001; 66(1):59-73
[12] Wax M B, Am J Ophthalmol. 1998; 125(2):145-157
[13] Romano C, Invest Ophthalmol Vis Sci. 1995; 36(10):1968-1975
[14] Tezel G, Arch Ophthalmol. 1999; 117(7):917-9248
[15] Maruyama I, Invest Ophthalmol Vis Sci. 2000; 41(7):1657-1665
[16] Yang J, Am J Ophthalmol. 2001; 131(4):421-426
[17] Yan J, Invest Ophthalmol Vis Sci. 2001; 42(6):1273-1276
[18] Tezel G, Invest Ophthalmol Vis Sci. 2001; 42(8):1787-1794
[19] Tezel G, J Neurosci. 2000; 20(23):8693-700
[20] Yuan L, Glia. 2000; 32(1):42-50
[21] Kremmer S, Clin Exp Immunol. 2001; 125(2):211-5
[22] Ricard C S, Exp Eye Res. 2001; 73(4):433-447
[23] Clark A F, FASEB J. 2001; 15(7):1251-1253
[24] Rezaie T, Science. 2002; 295(5557):1077-1079
[25] Surgucheva I, J Neurosci Res. 2002; 68(1):97-106
[26] Suguchov A, Cell Motil Cytoskeleton. 2001; 49(4):218-228
[27] Avrameas S, Immunol. Today 1991; 12:154-159
[28] Zimmermann C W, Eblen F, Herzau V, Reinauer K M, Graefe's Archive for Clin. and Exp. Opthalmology 1989; 227:521-525
[29] Zimmermann C W, Grus F H, Dux R, Electrophoresis 1995; 16:941-947
[30] Grus F H, Augustin A J, Toth-Sagi K, Koch F, Adv Ther. 1997; 14(1):8-13
[31] Grus F H, Augustin A J, Koch F, Zimmermann C W, Lutz J. Adv Ther. 1996; 13(4):203.

1. A method for the diagnosis of glaucoma, characterized in that in a first step, autoantibodies against ocular antigens are detected and measured in body fluids of an individual, and, in a second step, the autoantibody pattern is correlated with corresponding patterns of healthy individuals and glaucoma patients.

2. The method according to claim 1 wherein the ocular antigens are retinal antigens, optic nerve antigens, optic nerve head antigens, trabecular meshwork antigens, uveal antigens, or a mixture of such antigens.

3. The method according to claim 2 wherein the ocular antigens are retinal antigens or optic nerve head antigens or a mixture thereof.

4. The method according to claim 1 wherein the body fluid is serum, tears, saliva, urine, aqueous humour, or vitreous body of the eye.

5. The method according to claim 4 wherein the body fluid is serum or tears.

6. The method according to claim 4 wherein the body fluid is serum.

7. The method according to claim 1 wherein the autoantibody pattern consists of at least 10 autoantibodies.

8. The method according to claim 7 wherein the autoantibody pattern consists of at least 20 autoantibodies.

9. The method according to claim 7 wherein the autoantibody pattern consists of at least 30 autoantibodies.

10. The method according to claim 1 wherein the autoantibodies are detected and measured in a Western blot assay, chemiluminescence assay, ELISA, or RIA.

11. The method according to claim 10 wherein the autoantibodies are detected and measured in a Western blot assay.

12. The method according to claim 1 wherein the autoantibodies are detected and measured on a protein chip array using surface-enhanced laser desorption/ionization (SELDI) or matrix assisted laser desorption/ionization (MALDI) mass spectrometry techniques.

13. The method according to claim 12 wherein the autoantibodies are detected and measured on a protein chip array using surface-enhanced laser desorption/ionization (SELDI) mass spectrometry technique.

14. The method according to claim 1 wherein the autoantibodies are detected and measured by incubating protein-A chips with sera of individuals, treating said protein-A chips with a solution of ocular antigens, separating ocular antigens bound by autoantibodies on said protein-A chips by their molecular masses, and detecting separated ocular antigens by mass spectrometry.

15. The method according to claim 1 wherein the autoantibodies are detected and measured by binding autoantibodies in sera of individuals to beads, treating said beads with a solution of ocular antigens, eluting ocular antigens bound by antigen-antibody reaction from the beads, and analyzing eluted ocular antigens using SELDI-TOF or conventional electrophoretical techniques.

16. The method according to claim 1 wherein the technique to generate the autoantibody pattern is based on digital image detection, processing, and analysis.

17. The method according to claim 1 wherein autoantibodies are detected and measured in an individual's serum.

18. The method according to claim 17 wherein the change in the antibody pattern over time is used to assess the progression and/or severeness of glaucoma.

19. A method of comparison of complex autoantibody patterns by calculation wherein a pattern of autoantibodies against ocular antigens of an individual is compared with a pattern of autoantibodies against ocular antigens of healthy individuals and with a pattern of autoantibodies against ocular antigens of glaucoma patients.

20. The method of comparison according to claim 19 wherein the pattern of autoantibodies against ocular antigens of glaucoma patients is the autoantibody pattern of patients with primary open-angle glaucoma or of patients with normal tension glaucoma.

21. The method of comparison according to claim 19 wherein the calculation is based on artificial neural network technique.

22. A method for assessing an individual's risk for developing glaucoma with or without an elevated intraocular pressure, characterized in that in a first step, autoantibodies against ocular antigens are detected and measured in body fluids of the individual, and, in a second step, the autoantibody pattern is correlated with corresponding patterns of healthy individuals and of glaucoma patients.

23. A kit for the diagnosis of glaucoma according to claim 1, comprising a ready-to-use ocular antigen mixture and chemicals and materials needed to perform the biochemical analysis.

24. The kit according to claim 23 wherein the chemicals and materials are suitable for conventional Western blotting technique.

25. The kit according to claim 23 wherein the chemicals and materials are suitable for the SELDI-TOF technique.

26. The kit according to claim 23 comprising biochips.
